SEQUENCE LISTING (10 sequences)

SEQ ID NO: 1 - DNA, 20 bp, Artificial Sequence
  Chemically Synthesized
ctattccaccacggctggcg

SEQ ID NO: 2 - DNA, 20 bp, Artificial Sequence
  Chemically Synthesized
ctattccaccacggctggca

SEQ ID NO: 3 - DNA, 29 bp, Artificial Sequence
  Chemically Synthesized
agtggaatgaaaagtgcctttcacaggaa

SEQ ID NO: 4 - DNA, 21 bp, Artificial Sequence
  Chemically Synthesized
tgggaagaatgtttccagctc

SEQ ID NO: 5 - DNA, 20 bp, Artificial Sequence
  Chemically Synthesized
cattccttgggattggttac

SEQ ID NO: 6 - DNA, 20 bp, Artificial Sequence
  Chemically Synthesized
cattccttgggattggttat

SEQ ID NO: 7 - DNA, 27 bp, Artificial Sequence
  Chemically Synthesized
cctactcctattccaccacggctgtcg

SEQ ID NO: 8 - DNA, 27 bp, Artificial Sequence
  Chemically Synthesized
cctactcctattccaccacggctgtca

SEQ ID NO: 9 - DNA, 21 bp, Artificial Sequence
  Chemically Synthesized
gtcaccaatcccaaggaatga

SEQ ID NO: 10 - DNA, 21 bp, Artificial Sequence
  Chemically Synthesized
atcaccaatcccaaggaatga

(19) United States
(12) Patent Application Publication
(10) Pub. No.: US 2023/0265514 A1
(43) Pub. Date: Aug. 24, 2023

(54) RAPID CLINICAL TEST FOR GENETIC DIAGNOSIS INVOLVING KNOWN VARIANTS

(71) Applicant: YALE UNIVERSITY, New Haven, CT (US)

(72) Inventors: Curt Scharfe, New Haven, CT (US); Neeru Gandotra, New Haven, CT (US); Michael Murray, New Haven, CT (US)

(21) Appl. No.: 18/006,590

(22) PCT Filed: Jul. 27, 2021
(86) PCT No.: PCT/US2021/043271

§ 371 Date: Jan. 24, 2023

Related U.S. Application Data
(60) Provisional application No. 63/057,181, filed on Jul. 27, 2020.

(51) Int. Cl. C12Q 1/6883

(52) U.S. Cl. CPC C12Q 1/6883; C12Q 2600/156; C12Q 2600/118

(57) ABSTRACT

The present invention relates to a method of rapidly detecting genetic variation in individuals by PCR amplification of the locus of interest using allele-specific-oligonucleotides (ASOs), and visualizing the results. In particular, the present invention relates to the detection of genetic polymorphisms in the transthyretin (TTR) gene.

### CROSS REFERENCE TO RELATED APPLICATIONS

This application claims priority to U.S. Provisional Application No. 63/057,181, filed Jul. 27, 2020 which is hereby incorporated by reference herein in its entirety.

### BACKGROUND

The turn-around time for clinical genetic tests is typically on the order of days to weeks. Clinicians and patients will benefit from rapid and easy to interpret genetic testing within results available within the timeframe of an outpatient visit to a healthcare provider. This is especially true for relatively common and clinically important disease-causing genetic variants such as those associated with transthyretin (TTR) amyloidosis and others.

A “drop-in, drop-out” method for detection of TTR c.424G>A (Alexander et al., 2004, Mol Biotechnol. 2004 November;28(3):171-4) can be used for mutation screening, but cannot distinguish homozygous from heterozygous single-nucleotide variants (SNVs). Additional confirmation, for example using restriction enzyme digestion or sequencing would be required, which would increase the assay turn-around time and cost. Further, when the published primers were tested, the mutant primer was not specific for TTR c.424G>A. Another method uses the MaeIII restriction enzyme (Jacobson, 1992, Am J Hum Genet, 50(1):195-8), however this test is not specific because the restriction enzyme only cuts the wildtype and not the variant allele. In addition, this procedure is time consuming and cannot be used on blood.

Thus, there is a need in the art for compositions and methods for quick and accurate identification of disease-causing genetic variants. The present invention satisfies this need.

### SUMMARY OF THE INVENTION

In one embodiment, the invention relates to a system comprising at least one set of primers for nucleic acid amplification, comprising: at least one variant allele-specific primer, wherein the 3′ nucleotide of the variant allele-specific primer is a nucleotide that is the first allelic variant of a target sequence or a nucleotide that is complementary to the first allelic variant of a target sequence, wherein at least one nucleotide at a position of 5, 4, 3 or 2 bases from the 3′end of the primer is not complementary to the target sequence, and wherein the variant allele-specific primer comprises at least one modified linkage; and at least one locus-specific primer that is complementary to a region of the target sequence that is upstream or downstream from the first allelic variant and on the opposite strand.

In one embodiment, the nucleotide at the position 3 bases from the 3′end of the variant allele-specific primer is not complementary to the target sequence.

In one embodiment, at least one primer comprises at least one phosphorothioate linkage. In one embodiment, at least one primer comprises at least three phosphorothioate linkages.

In one embodiment, the system further comprises at least one reference allele-specific primer, wherein the 3′ nucleotide of the reference allele-specific primer is a nucleotide that is the wild-type or reference nucleotide of a target sequence or a nucleotide that is complementary to the wild-type or reference nucleotide of a target sequence, wherein at least one nucleotide at a position of 5, 4, 3 or 2 bases from the 3′end of the primer is not complementary to the target sequence, and wherein the reference allele-specific primer comprises at least one modified linkage.

In one embodiment, the nucleotide at the position 3 bases from the 3′end of the reference allele-specific primer is not complementary to the target sequence.

In one embodiment, the reference allele-specific primer comprises at least one phosphorothioate linkage. In one embodiment, at least one primer comprises at least three phosphorothioate linkages.

In one embodiment, the system comprises at least two variant allele-specific primers, wherein the at least two variant allele-specific primers are specific for the same variant allele, wherein at least one variant allele-specific primer is a forward primer comprising a nucleotide that is the first allelic variant of a target sequence and at least one variant allele-specific primer is reverse primer comprising a nucleotide that is complementary to the first allelic variant of the target sequence; and at least two locus-specific primers.

In one embodiment, at least one allele-specific primer is specific for a transthyretin 424G>A variant.

In one embodiment, at least one allele-specific primer is SEQ ID NO:2 or SEQ ID NO:6.

In one embodiment, at least one reference allele-specific primer is SEQ ID NO:1 or SEQ ID NO:5.

In one embodiment, at least one locus specific primer is SEQ ID NO:3 or SEQ ID NO:4.

In one embodiment, the invention relates to a method of identifying a subject as having a genetic variant allele comprising: a) contacting a nucleic acid sample of the subject with a system comprising at least one set of primers for nucleic acid amplification, comprising: at least one variant allele-specific primer, wherein the 3′ nucleotide of the variant allele-specific primer is a nucleotide that is the first allelic variant of a target sequence or a nucleotide that is complementary to the first allelic variant of a target sequence, wherein at least one nucleotide at a position of 5, 4, 3 or 2 bases from the 3′end of the primer is not complementary to the target sequence, and wherein the variant allele-specific primer comprises at least one modified linkage; and at least one locus-specific primer that is complementary to a region of the target sequence that is upstream or downstream from the first allelic variant and on the opposite strand, b) amplifying a target nucleotide sequence in the nucleic acid sample; and c) detecting an amplification product.

In one embodiment, the method comprises contacting the sample with at least two sets of primers in a single reaction, wherein amplification with the at least two sets of primers generates amplification products with distinct sizes.

In one embodiment, the sample is a blood sample.

In one embodiment, the invention relates to a method of identifying a subject as being heterozygous or homozygous for a genetic variant allele comprising: a) contacting a nucleic acid sample of the subject with a system comprising at least one set of primers for nucleic acid amplification, comprising: at least one variant allele-specific primer, wherein the 3′ nucleotide of the variant allele-specific primer is a nucleotide that is the first allelic variant of a target sequence or a nucleotide that is complementary to the first allelic variant of a target sequence, wherein at least one nucleotide at a position of 5, 4, 3 or 2 bases from the 3′end of the primer is not complementary to the target sequence, and wherein the variant allele-specific primer comprises at least one modified linkage; and at least one locus-specific primer that is complementary to a region of the target sequence that is upstream or downstream from the first allelic variant and on the opposite strand and wherein the system further comprises at least one reference allele-specific primer, wherein the 3′ nucleotide of the reference allele-specific primer is a nucleotide that is the wild-type or reference nucleotide of a target sequence or a nucleotide that is complementary to the wild-type or reference nucleotide of a target sequence, wherein at least one nucleotide at a position of 5, 4, 3 or 2 bases from the 3′end of the primer is not complementary to the target sequence, and wherein the reference allele-specific primer comprises at least one modified linkage, b) amplifying a target nucleotide sequence in the nucleic acid sample; and c) detecting one or more amplification product, wherein detection of an amplification product upon amplification with at least one variant allele specific primer and detection of an amplification product upon amplification with at least one reference allele specific primer is an indicator that the subject is heterozygous for the variant allele, whereas detection of an amplification product upon amplification with at least one variant allele specific primer but not upon amplification with at least one reference allele specific primer is an indicator that the subject is homozygous for the variant allele.

In one embodiment, the genetic variant allele is a disease-associated variant allele.

In one embodiment, the genetic variant allele is a transthyretin 424G>A variant allele.

In one embodiment, the method further comprises diagnosing the subject as having an increased risk of developing transthyretin (TTR) amyloidosis, senile systemic amyloidosis (SSA), familial amyloid polyneuropathy (FAP), or familial amyloid cardiomyopathy (FAC) based upon identification that the subject is heterozygous or homozygous for the transthyretin 424G>A variant allele.

In one embodiment, the method comprises contacting the sample with at least two sets of primers in a single reaction, wherein amplification with the at least two sets of primers generates amplification products with distinct sizes.

In one embodiment, the sample is a blood sample.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts the primer design.

FIG. 2 depicts the results of an exemplary TTR assay performed on genomic DNA samples. The data for the samples and primers evaluated in this figure are provided in Table 2.

FIG. 3 depicts the results of an exemplary TTR assay performed on pinprick blood samples. The data for the samples and primers evaluated in this figure are provided in Table 2.

FIG. 4 depicts the results of an exemplary TTR assay performed on patient blood samples. The data for the samples and primers evaluated in this figure are provided in Table 2.

FIG. 5A and FIG. 5B depict exemplary sequencing traces which confirms that the patient is homozygous for V122I variant (G>A). FIG. 5A depicts a sequence trace of a control sample showing homozygous wild type allele(G). FIG. 5B depicts a sequence trace of a control sample showing the homozygous variant allele (A). Sanger confirmation was done using primers in both directions.

FIG. 6 depicts the results of an exemplary TTR assay performed on a Nik-2 patient sample with the 200 bp primer set. The data for the sample evaluated in this figure are provided in Table 4. This patient is heterozygous for V122I.

FIG. 7 depicts the results of an exemplary TTR assay performed on a Nik-2 patient sample with the 538 bp primer set. The data for the sample evaluated in this figure are provided in Table 4. This patient is heterozygous for V122I.

FIG. 8A and FIG. 8B depict exemplary sequencing traces which confirms that the Nik-2 patient is heterozygous for V122I variant (G>A). FIG. 8A depicts a sequence trace of the forward strand with the 293-F forward primer. FIG. 8B depicts a sequence trace of the reverse strand with the OR-R reverse primer.

### DETAILED DESCRIPTION

The present invention provides a method for detecting variants in disease-associated genes. The present method allows for the identification of subjects as homozygous or heterozygous for a specific variation as compared to a reference or wild-type gene. The invention also provides a kit containing oligonucleotides necessary to detect the presence of genetic variants.

In one embodiment, the invention provides allele specific oligonucleotide (ASO) primers for the identification of a V142I variant in the human transthyretin (TTR) gene.

In one embodiment, the invention provides methods for detecting the presence of a TTR variant in a patient sample. In one embodiment, the sample is a pinprick blood sample.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice of and/or for the testing of the present invention, the preferred materials and methods are described herein. In describing and claiming the present invention, the following terminology will be used according to how it is defined, where a definition is provided.

It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which the invention pertains. Although any methods and materials similar or equivalent to those described herein can be used in the practice of and/or for the testing of the present invention, the preferred materials and methods are described herein. In describing and claiming the present invention, the following terminology will be used according to how it is defined, where a definition is provided.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

The term “allele” refers to any of the forms of the same gene that occur at the same locus on a homologous chromosome but differ in base sequence.

“Amplification reaction mixture” refers to an aqueous solution comprising the various reagents used to amplify a target nucleic acid. These include: enzymes, aqueous buffers, salts, target nucleic acid, and deoxynucleoside triphosphates. Depending upon the context, the mixture can be either a complete or incomplete amplification reaction mixture.

“Amplification reaction system” refers to any in vitro means for multiplying the copies of a target sequence of nucleic acid. Such methods include, but are not limited to, polymerase chain reaction amplification (PCR), DNA ligase, QB RNA replicase, and RNA transcription-based amplification systems. These involve multiple amplification reagents and are more fully described below.

“Amplification reaction tube(s)” refers to a container suitable for holding the amplification reagents. Generally, the tube is constructed of inert components so as to not inhibit or interfere with the amplification system being used. Where the system requires thermal cycling of repeated heating and cooling, the tube must be able to withstand the cycling process and, typically, precisely fit the wells of the thermocycler.

“Amplification reagents” refer to the various buffers, enzymes, primers, deoxynucleoside triphosphates (both conventional and unconventional), and primers used to perform the selected amplification procedure.

“Amplifying” or “Amplification”, which typically refers to an “exponential” increase in target nucleic acid, is being used herein to describe both linear and exponential increases in the numbers of a select target sequence of nucleic acid.

The term “complementary” (or “complementarity”) refers to the specific base pairing of nucleotide bases in nucleic acids within a contiguous region of double stranded nucleic acid, such as between an ASO sequence and its complementary sequence in a target polynucleotide.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared X 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Hybridizing” refers the binding of two single stranded nucleic acids via complementary base pairing.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Nucleotide polymerases” refers to enzymes able to catalyze the synthesis of DNA or RNA from nucleoside triphosphate precursors. In the amplification reactions of this invention, the polymerases are template-dependent and typically add nucleotides to the 3′-end of the polymer being formed. It is most preferred that the polymerase is thermostable as described in U.S. Pat. Nos. 4,889,818 and 5,079,352.

The terms “patient,” “subject,” “individual,” and the like are used interchangeably herein, and refer to any animal, or cells thereof whether in vitro or in situ, amenable to the methods described herein. In some embodiments, the patient, subject or individual is a mammal such as a non-primate (e.g., cows, pigs, horses, cats, dogs, rats, etc.) and a primate (e.g., monkey and human), most preferably a human. In certain non-limiting embodiments, the patient, subject or individual is a human.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning and amplification technology, and the like, and by synthetic means. An “oligonucleotide” as used herein refers to a short polynucleotide, typically less than 100 bases in length.

“Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “primer” refers to an oligonucleotide, whether natural or synthetic, capable of acting as a point of initiation of DNA synthesis under conditions in which synthesis of a primer extension product complementary to a nucleic acid strand is induced, i.e., in the presence of four different nucleoside triphosphates and an agent for polymerization (i.e., DNA polymerase or reverse transcriptase) in an appropriate buffer and at a suitable temperature. A primer is preferably a single-stranded oligodeoxyribonucleotide. The appropriate length of a primer depends on the intended use of the primer but typically ranges from 15 to 25 nucleotides. Short primer molecules generally require cooler temperatures to form sufficiently stable hybrid complexes with the template. A primer need not reflect the exact sequence of the template but must be sufficiently complementary to hybridize with a template.

The term “primer” may refer to more than one primer, particularly in the case where there is some ambiguity in the information regarding one or both ends of the target region to be amplified. For instance, if a region shows significant levels of polymorphism in a population, mixtures of primers can be prepared that will amplify alternate sequences. A primer can be labeled, if desired, by incorporating a label detectable by spectroscopic, photochemical, biochemical, immunochemical, or chemical means. For example, useful labels include 32 P, fluorescent dyes, electron-dense reagents, enzymes (as commonly used in an ELISA), biotin, or haptens and proteins for which antisera or monoclonal antibodies are available. A label can also be used to “capture” the primer, so as to facilitate the immobilization of either the primer or a primer extension product, such as amplified DNA, on a solid support.

The term “isolated” when used in relation to a nucleic acid, as in “isolated oligonucleotide” or “isolated polynucleotide” refers to a nucleic acid sequence that is identified and separated from at least one contaminant with which it is ordinarily associated in its source. Thus, an isolated nucleic acid is present in a form or setting that is different from that in which it is found in nature. In contrast, non-isolated nucleic acids (e.g., DNA and RNA) are found in the state they exist in nature. For example, a given DNA sequence (e.g., a gene) is found on the host cell chromosome in proximity to neighboring genes; RNA sequences (e.g., a specific mRNA sequence encoding a specific protein), are found in the cell as a mixture with numerous other mRNAs that encode a multitude of proteins. However, isolated nucleic acid includes, by way of example, such nucleic acid in cells ordinarily expressing that nucleic acid where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide contains at a minimum, the sense or coding strand (i.e., the oligonucleotide may be single-stranded), but may contain both the sense and anti-sense strands (i.e., the oligonucleotide may be double-stranded).

The term “position” refers to a defined site on a nucleic acid molecule. Such a position may, for example, be occupied by a nucleotide.

A “therapeutic” treatment is a treatment administered to a subject who exhibits signs of pathology, for the purpose of diminishing or eliminating those signs.

As used herein, “treating a disease or disorder” means reducing the frequency with which a symptom of the disease or disorder is experienced by a patient. Disease and disorder are used interchangeably herein.

The phrase “therapeutically effective amount,” as used herein, refers to an amount that is sufficient or effective to prevent or treat (delay or prevent the onset of, prevent the progression of, inhibit, decrease or reverse) a disease or condition, including alleviating symptoms of such diseases.

To “treat” a disease as the term is used herein, means to reduce the frequency or severity of at least one sign or symptom of a disease or disorder experienced by a subject.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

To increase the efficiency and facilitate identification of variants in samples, a combined approach including PCR amplification and allele-specific oligonucleotide (ASO) hybridization was developed. The assay involves multiple ASOs for TTR V122I, designed in both the forward and reverse direction and at two different lengths (e.g., 200 bp and 538 bp or 200 bp and 293 bp). This assay enables detection of both homozygous (wildtype or variant) or heterozygous TTR variants, creates redundancy for primer failures, enables primer multiplexing, and increases assay speed.

ASO primers according to the invention can be designed to be specific for amplification of any wild type or variant allele of interest. In one embodiment, the variant allele is a disease-associated variant. Disease-associated variant alleles having clinical significance for which ASO primers can be designed, include, but are not limited to, those identified in the ClinVar database (ncbi.nlm.nih.gov/clinvar) as risk factors, pathogenic, likely pathogenic, having uncertain significance, likely benign, or benign. Exemplary disease-associated genetic variants for which ASO primers of the invention can be designed include, but are not limited to, TTR c.424G>A (p.Val142Ile), TTR c.148G>A (p.Val50Met), rs10757278(G shows an increased association for myocardial infarctions), and CFTR c.1520_1522delTCT.

ASO primers according to the invention can be designed to detect any type of variation, including, but not limited to a deletion, duplication, indel, insertion, single nucleotide polymorphism (SNP), known variant, and de-novo variant, such that one or more discrete primer set can be designed specific for the variation. In one embodiment, at least two primer sets are designed for detection of each variation to create redundancy for primer failures, and reduce false positive and false negative results. In one embodiment, amplification of a target nucleotide sequence using at least two primer sets results in generation of amplification products having distinct sizes.

This approach has a potential for automation through the use of the 96-well microplates and robotic workstations for high sample throughput. A PCR-ASO assay is simple, efficient and cost effective, particularly when a large number of samples are to be screened for several DNA variants.

Components of the assay systems described herein may be conveniently packaged in kits. Such kits may contain, for example, various reagents for individual assays and instructions for their use. Typical kit components include, for example, (1) one or more ASO primers for amplification of specific variant alleles; (2) one or more ASO primers for amplification of specific reference alleles; and (3) reagents for PCR amplification.

### ASO Primers

Allele-specific amplification utilizing oligonucleotides complementary to either a wild-type or variant sequence, are included in the present invention as methods for identifying specific mutations. For example, oligonucleotides can be designed such that they specifically hybridize to a wild-type or variant nucleotide sequence in a polynucleotide. The oligonucleotides support amplification when hybridized to the appropriate complementary sequence.

In one embodiment, the invention provides ASO primers for detection of genetic variants. In one embodiment, the invention provides systems comprising multiple ASO primers, wherein at least one primer is specific for the wild-type allele and at least one primer is specific for a genetic variant. In one embodiment, at least one of the forward and reverse primers of the invention are designed to have one or more phosphorothioate bond modification at position 1, 2, 3, 4, 5 or 6 from the 3′ base of each ASO primer. In one embodiment, at least one of the forward and reverse primers have 1, 2, 3, 4, or 5 phosphorothioate bond modification at position 1, 2, 3, 4, 5 or 6 from the 3′ base of each ASO primer. In one embodiment, at least one of the forward and reverse primers have 3 phosphorothioate bond modifications at positions 1, 2, 3, and 4 from the 3′ base of the primer. In one embodiment, at least one of the forward and reverse primers have 4 phosphorothioate bond modifications at positions 1, 2, 3,and 4 from the 3′ base of the primer. In one embodiment the forward primer comprises 3 phosphorothioate bond modifications at positions 1, 2, and 3 from the 3′ base of the primer, and the reverse primer comprises 4 phosphorothioate bond modifications at positions 1, 2, 3, and 4 from the 3′ base of the primer.

In one embodiment, at least one primer contains a mismatch to the target sequence to increase the allele specificity of the oligonucleotide in both the WT and the variant oligonucleotide. In one embodiment, the mismatch is at the third base from the 3′ end of the ASO.

In general, the primers used according to the method of the invention embrace oligonucleotides of sufficient length and appropriate sequence which provides specific initiation of polymerization of a significant number of nucleic acid molecules containing the target nucleic acid under the conditions of stringency for the reaction utilizing the primers. In this manner, it is possible to selectively amplify the specific target nucleic acid sequence containing the nucleic acid of interest. Specifically, the term “primer” as used herein refers to a sequence comprising two or more deoxyribonucleotides or ribonucleotides, preferably at least eight, which sequence is capable of initiating synthesis of a primer extension product that is substantially complementary to a target nucleic acid strand. The oligonucleotide primer typically contains 15-22 or more nucleotides, although it may contain fewer nucleotides as long as the primer is of sufficient specificity to allow essentially only the amplification of the specifically desired target nucleotide sequence (i.e., the primer is substantially complementary).

Experimental conditions conducive to synthesis include the presence of nucleoside triphosphates and an agent for polymerization, such as DNA polymerase, and a suitable temperature and pH. In one embodiment, the primer is single stranded for maximum efficiency in amplification, but may be double stranded. If double stranded, the primer is first treated to separate its strands before being used to prepare extension products. In one embodiment, the primer is an oligodeoxyribonucleotide. The primer must be sufficiently long to prime the synthesis of extension products in the presence of the inducing agent for polymerization. The exact length of primer will depend on many factors, including temperature, buffer, and nucleotide composition.

Primers used according to the method of the invention are designed to be “substantially” complementary to each strand of mutant nucleotide sequence to be amplified. Substantially complementary means that the primers must be sufficiently complementary to hybridize with their respective strands under conditions which allow the agent for polymerization to function. In other words, the primers should have sufficient complementarily with the flanking sequences to hybridize therewith and permit amplification of the target nucleotide sequence. In one embodiment, the 3′ terminus of the primer that is extended has perfectly base paired complementarity with the complementary flanking strand. In one embodiment, the primer contains a mismatch to the target sequence, which functions to increase the allele specificity of the oligonucleotide. In one embodiment, the mismatch is 5, 4, 3 or 2 bases from the 3′end of the primer. In one embodiment, the mismatch is 3 bases from the 3′end of the primer.

Oligonucleotide primers used according to the invention are employed in any amplification process that produces increased quantities of target nucleic acid. Typically, one primer is complementary to the negative (−) strand of the mutant nucleotide sequence and the other is complementary to the positive (+) strand. Annealing the primers to denatured nucleic acid followed by extension with an enzyme, such as the large fragment of DNA. Polymerase I (Klenow) or Taq DNA polymerase and nucleotides or ligases, results in newly synthesized + and − strands containing the target nucleic acid. Because these newly synthesized nucleic acids are also templates, repeated cycles of denaturing, primer annealing, and extension results in exponential production of the region (i.e., the target mutant nucleotide sequence) defined by the primer. The product of the amplification reaction is a discrete nucleic acid duplex with termini corresponding to the ends of the specific primers employed. Those of skill in the art will know of other amplification methodologies which can also be utilized to increase the copy number of target nucleic acid.

The oligonucleotide primers for use in the invention may be prepared using any suitable method, such as conventional phosphotriester and phosphodiester methods or automated embodiments thereof. In one such automated embodiment, diethylphosphoramidites are used as starting materials and may be synthesized as described by Beaucage, et al. (Tetrahedron Letters, 22:1859-1862, 1981). One method for synthesizing oligonucleotides on a modified solid support is described in U.S. Pat. No. 4,458,066. One method of amplification which can be used according to this invention is the polymerase chain reaction (PCR) described in U.S. Pat. Nos. 4,683,202 and 4,683,195.

A nucleic acid primer of the present invention will generally contain phosphodiester bonds, although in some cases, as outlined below, nucleic acid analogs are included that may have alternate backbones, comprising, for example, phosphoramide (Beaucage et al. (1993) Tetrahedron 49(10):1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 1419), phosphorothioate (Mag et al. (1991) Nucleic Acids Res. 19:1437; and U.S. Pat. No. 5,644,048), phosphorodithioate (Briu et al. (1989) J. Am. Chem. Soc. 111:2321, O-methylphosphoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp 169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments.

In certain embodiments phosphodiester, phosphorothioate and/or other modified linkages are used. In one embodiment, a phosphorothioate linkage is included between at least one of the 1-5 nucleotides at the 3′ end or the 5′ end to prevent exonuclease degradation. In one embodiment, a phosphorothioate linkage is included to link the base at the 3′ end of the oligonucleotide to the oligonucleotide. In one embodiment, one or more phosphorothioate linkage are included between at least one of nucleotides 1-2, 2-3, 3-4, and 4-5 at the 3′ end of the primer. In one embodiment, 3 phosphorothioate linkages are included between at least three of nucleotides 1-2, 2-3, 3-4, and 4-5 at the 3′ end of the primer. In one embodiment, 4 phosphorothioate linkages are included between nucleotides 1-2, 2-3, 3-4, and 4-5 at the 3′ end of the primer.

### ASO Assay

In one embodiment, the primers of the invention are used in conjunction with a polymerase chain reaction (PCR) amplification of the target nucleic acid. Although the PCR process is well known in the art (see U.S. Pat. Nos. 4,683,195; 4,683,202; and 4,965,188, each of which is incorporated herein by reference) and although commercial vendors, such as Perkin Elmer, sell PCR reagents manufactured and developed by Hoffmann-La Roche and publish PCR protocols, some general PCR information is provided below for purposes of clarity and full understanding of the invention for those unfamiliar with the PCR process.

To amplify a target nucleic acid sequence in a sample by PCR, the sequence must be accessible to the components of the amplification system. In general, this accessibility is ensured by isolating the nucleic acids from the sample. A variety of techniques for extracting ribonucleic acids from biological samples are known in the art. For example, see those described in Rotbart et al., 1989, in PCR Technology (Erlich ed., Stockton Press, New York) and Han et al. 1987, Biochemistry 26:1617-1625. Alternatively, if the sample is fairly readily disruptable, the nucleic acid need not be purified prior to amplification by the PCR technique, i.e., if the sample is comprised of cells, particularly peripheral blood lymphocytes or monocytes, lysis and dispersion of the intracellular components may be accomplished merely by suspending the cells in hypotonic buffer.

The first step of each cycle of the PCR involves the separation of the nucleic acid duplex. Of course, if the target nucleic acid is single- stranded, i.e., single-stranded DNA or RNA, no initial separation step is required during the first cycle. Once the strands are separated, the next step in PCR involves hybridizing the separated strands with primers that flank the target sequence. The primers are then extended to form complementary copies of the target strands. For successful PCR amplification, the primers are designed so that the position at which each primer hybridizes along a duplex sequence is such that an extension product synthesized from one primer, when separated from the template (complement), serves as a template for the extension of the other primer. The cycle of denaturation, hybridization, and extension is repeated as many times as necessary to obtain the desired amount of amplified nucleic acid.

In one embodiment of the PCR process, strand separation is achieved by heating the reaction to a sufficiently high temperature for a sufficient time to cause the denaturation of the duplex but not to cause an irreversible denaturation of the polymerase (see U.S. Pat. No. 4,965,188). Typical heat denaturation involves temperatures ranging from about 80° C. to 105° C. for times ranging from seconds to minutes. Strand separation, however, can be accomplished by any suitable denaturing method including physical, chemical, or enzymatic means. Strand separation may be induced by a helicase, for example, or an enzyme capable of exhibiting helicase activity. For example, the enzyme RecA has helicase activity in the presence of ATP. The reaction conditions suitable for strand separation by helicases are known in the art (see Kuhn Hoffman-Berling, 1978, CSH-Quantitative Biology 43:63-67; and Radding, 1982, Ann. Rev. Genetics 16:405-436).

Template-dependent extension of primers in PCR is catalyzed by a polymerizing agent in the presence of adequate amounts of four deoxyribonucleoside triphosphates (typically dATP, dGTP, dCTP, and dTTP) in a reaction medium comprised of the appropriate salts, metal cations, and pH buffering system. Suitable polymerizing agents are enzymes known to catalyze template-dependent DNA synthesis. In one embodiment, the initial template for primer extension is RNA. Polymerizing agents suitable for synthesizing a complementary, copy-DNA (cDNA) sequence from the RNA template are reverse transcriptase (RT), such as avian myeloblastosis virus RT, Moloney murine leukemia virus RT, or Thermus thermophilus (Tth) DNA polymerase, a thermostable DNA polymerase with reverse transcriptase activity marketed by Perkin Elmer. Typically, the genomic RNA/cDNA duplex template is heat denatured during the first denaturation step after the initial reverse transcription step leaving the DNA strand available as an amplification template. Suitable polymerases for use with a DNA template include, for example, E. coli DNA polymerase I or its Klenow fragment, T4 DNA polymerase, Tth polymerase, and Taq polymerase, a heat-stable DNA polymerase isolated from Thermus aquaticus and high fidelity polymerases as discussed elsewhere herein.

When RNA is amplified, an initial reverse transcription (RT) step is carried out to create a DNA copy (cDNA) of the RNA. PCT patent publication No. WO 91/09944, published Jul. 11, 1991, incorporated herein by reference, describes high-temperature reverse transcription by a thermostable polymerase that also functions in PCR amplification. High-temperature RT provides greater primer specificity and improved efficiency. Copending U.S. patent application Ser. No. 07/746,121, filed Aug. 15, 1991, incorporated herein by reference, describes a “homogeneous RTPCR” in which the same primers and polymerase suffice for both the reverse transcription and the PCR amplification steps, and the reaction conditions are optimized so that both reactions occur without a change of reagents. Thermus thermophilus DNA polymerase, a thermostable DNA polymerase that can function as a reverse transcriptase, is used for all primer extension steps, regardless of template. Both processes can be done without having to open the tube to change or add reagents; only the temperature profile is adjusted between the first cycle (RNA template) and the rest of the amplification cycles (DNA template).

The target nucleic acid molecule may be, for example, DNA or RNA, including messenger RNA (mRNA), wherein DNA or RNA may be single stranded or double stranded. In the event that RNA is to be used as a template, enzymes, and/or conditions optimal for reverse transcribing the template to DNA would be utilized. In addition, a DNA-RNA hybrid which contains one strand of each may be utilized. A mixture of nucleic acids may also be employed, or the nucleic acids produced in a previous amplification reaction herein, using the same or different primers may be so utilized. The nucleotide sequence to be amplified may be a fraction of a larger molecule or can be present initially as a discrete molecule, such that the specific sequence constitutes the entire nucleic acid. It is not necessary that the sequence to be amplified be present initially in a pure form; it may be a minor fraction of a complex mixture, such as contained in whole genomic DNA.

Where the target nucleotide sequence of the sample contains two strands, it is necessary to separate the strands of the nucleic acid before it can be used as the template. Strand separation can be effected either as a separate step or simultaneously with the synthesis of the primer extension products. This strand separation can be accomplished using various suitable denaturing conditions, including physical, chemical, or enzymatic means; the word “denaturing” includes all such means. One physical method of separating nucleic acid strands involves heating the nucleic acid until it is denatured. Typical heat denaturation may involve temperatures ranging from about 80° to 105° C. for times ranging from about 1 to 10 minutes. Strand separation may also be induced by an enzyme from the class of enzymes known as helicases or by the enzyme RecA, which has helicase activity, and in the presence of riboATP which is known to denature DNA. The reaction conditions suitable for strand separation of nucleic acids with helicases are described by Kuhn Hoffmann-Berling (CSH-Quantitative Biology, 43:63, 1978) and techniques for using RecA are reviewed in C. Radding (Ann. Rev. Genetics, 16:405-437, 1982).

If the nucleic acid containing the target nucleic acid to be amplified is single stranded, its complement is synthesized by adding one or two oligonucleotide primers. If a single primer is utilized, a primer extension product is synthesized in the presence of primer, an agent for polymerization, and the four nucleoside triphosphates described below. The product will be complementary to the single-stranded nucleic acid and will hybridize with a single-stranded nucleic acid to form a duplex of unequal length strands that may then be separated into single strands to produce two single separated complementary strands. Alternatively, two primers may be added to the single-stranded nucleic acid and the reaction carried out as described.

When complementary strands of nucleic acid or acids are separated, regardless of whether the nucleic acid was originally double or single stranded, the separated strands are ready to be used as a template for the synthesis of additional nucleic acid strands. This synthesis is performed under conditions allowing hybridization of primers to templates. In one embodiment, synthesis occurs in a buffered aqueous solution, having a pH of 7-9. In one embodiment, a molar excess (for example, at least 10:1 primer:template) of the two oligonucleotide primers is added to the buffer containing the separated template strands. It is understood, however, that the amount of complementary strand may not be known if the process of the invention is used for diagnostic applications, so that the amount of primer relative to the amount of complementary strand cannot be determined with certainty. As a practical matter, however, the amount of primer added will generally be in molar excess over the amount of complementary strand (template) when the sequence to be amplified is contained in a mixture of complicated long-chain nucleic acid strands. A large molar excess is preferred to improve the efficiency of the process.

In some amplification embodiments, the substrates, for example, the deoxyribonucleotide triphosphates dATP, dCTP, dGTP, and dTTP, are added to the synthesis mixture, either separately or together with the primers, in adequate amounts and the resulting solution is heated to about 90°-100° C. from about 1 to 10 minutes, preferably from 1 to 4 minutes. After this heating period, the solution is allowed to cool to room temperature, which is preferable for the primer hybridization. To the cooled mixture is added an appropriate agent for effecting the primer extension reaction (called herein “agent for polymerization”), and the reaction is allowed to occur under conditions known in the art. The agent for polymerization may also be added together with the other reagents if it is heat stable. This synthesis (or amplification) reaction may occur at room temperature up to a temperature above which the agent for polymerization no longer functions. Thus, for example, if DNA polymerase is used as the agent, the temperature is generally no greater than about 40° C.

The agent for polymerization may be any compound or system which will function to accomplish the synthesis of primer extension products, including enzymes. Suitable enzymes for this purpose include, for example, E. coli DNA polymerase I, Taq polymerase, Klenow fragment of E. coli DNA polymerase I, T4 DNA polymerase, other available DNA polymerase, polymerase muteins, reverse transcriptase, ligase, and other enzymes, including heat-stable enzymes (i.e., those enzymes which perform primer extension after being subjected to temperatures sufficiently elevated to cause denaturation). In one embodiment, the polymerase for use in the method of the invention is a high fidelity polymerase. Exemplary high fidelity polymerases include, but are not limited to Q5 high fidelity polymerase, Phusion High-Fidelity DNA polymerase, Platinum SuperFi DNA polymerase, PfuUltra High-Fidelity DNA polymerase, HotStar HiFidelity DNA polymerase, and KAPA HiFi polymerase. In one embodiment, the polymerase for use in the method of the invention comprises 3′to 5′ exonuclease activity. In one embodiment, an enzyme comprising 3′to 5′ exonuclease activity is included in the reaction with a polymerase. For example, in one embodiment, high fidelity polymerization is provided by a mixture of Platinum® Taq DNA Polymerase and the proofreading enzyme Pyrococcus species GB-D polymerase. Suitable enzymes will facilitate combination of the nucleotides in the proper manner to form the primer extension products which are complementary to each mutant nucleotide strand. Generally, the synthesis will be initiated at the 3′ end of each primer and proceed in the 5′ direction along the template strand, until synthesis terminates, producing molecules of different lengths. There may be agents for polymerization, however, which initiate synthesis at the 5′ end and proceed in the other direction, using the same process as described above. In any event, the method of the invention is not to be limited to the embodiments of amplification which are described herein.

The newly synthesized mutant nucleotide strand and its complementary nucleic acid strand will form a double-stranded molecule under hybridizing conditions described above and this hybrid is used in subsequent steps of the process. In the next step, the newly synthesized double-stranded molecule is subjected to denaturing conditions using any of the procedures described above to provide single-stranded molecules.

The above process is repeated on the single-stranded molecules. Additional agent for polymerization, nucleosides, and primers may be added, if necessary, for the reaction to proceed under the conditions prescribed above. Again, the synthesis will be initiated at one end of each of the oligonucleotide primers and will proceed along the single strands of the template to produce additional nucleic acid. After this step, half of the extension product will consist of the specific nucleic acid sequence bounded by the two primers.

The steps of denaturing and extension product synthesis can be repeated as often as needed to amplify the target mutant nucleotide sequence to the extent necessary for detection. The amount of the mutant nucleotide sequence produced will accumulate in an exponential fashion.

The target nucleic acid sequence of the invention can be derived from any organism or subject including mouse, rat, cow, pig, human, horse, sheep, goat, chicken, turkey, fish and other species are also included herein. Screening procedures which rely on nucleic acid hybridization make it possible to isolate any gene sequence from any organism, provided the appropriate probe is available. Oligonucleotide probes, which correspond to a part of the sequence encoding the protein in question, can be synthesized chemically. This requires that short, oligopeptide stretches of amino acid sequence must be known. The DNA sequence encoding the protein can be deduced from the genetic code, however, the degeneracy of the code must be taken into account. It is possible to perform a mixed addition reaction when the sequence is degenerate. This includes a heterogeneous mixture of denatured double-stranded DNA. For such screening, hybridization is preferably performed on either single-stranded DNA or denatured double-stranded DNA. Hybridization is particularly useful in the detection of cDNA clones derived from sources where an extremely low amount of mRNA sequences relating to the polypeptide of interest are present. In other words, by using stringent hybridization conditions directed to avoid non-specific binding, it is possible, for example, to allow the autoradiographic visualization of a specific cDNA clone by the hybridization of the target DNA to that single probe in the mixture which is its complete complement (Wallace, et al, Nucl. Acid Res. 9:879, 1981).

TTR Allelic Variants The method of the invention includes identifying allelic variants in a subject. The subject may be homozygous or heterozygous for a TTR variant. As used herein, an “allele” is a gene present in more than one form (different sequence) in a genome. “Homozygous”, according to the present invention, indicates that the TTR gene is present as two copies (i.e., alleles), each allele being identical in sequence and function to the other allele. For example, a subject homozygous for the wild-type TTR gene contains at least two copies of the TTR wild-type sequence. Such a subject would not be predisposed to an increase in muscle mass and, therefore, would not exhibit the “double-muscling” phenotype. In contrast, a subject heterozygous or homozygous for a variant TTR allele, such as, for example, the TTR allele of the invention contained in Belgian Blue cattle, contains at least one variant allele of the TTR gene and would exhibit the double-muscling phenotype or at least be predisposed to the phenotype. Subjects that carry at least one variant V122I TTR allele may develop cardiac amyloidosis at some later age (e.g., 60 or older.) Homozygous carriers of the variant V122I TTR allele may develop the disease earlier, (e.g., 50 or older.)

“Heterozygous” as used in the present invention, indicates that one copy of the wild-type allele and one copy of the variant allele are present in the genome. A subject having such a genome is heterozygous. Heterozygous, as used in the present invention, also encompasses a subject having two different mutations in TTR alleles. For example, a subject carrying two variant alleles, each of which are variant at the same nucleotide position, would be heterozygous for two different TTR variants.

A heterozygous subject or a subject who is homozygous for a TTR variant may be a carrier for, or have an increased risk of, developing transthyretin amyloidosis or a disease or disorder associated therewith. Diseases associated with altered levels or function of the TTR gene, include, but are not limited to, senile systemic amyloidosis (SSA), familial amyloid polyneuropathy (FAP), and familial amyloid cardiomyopathy (FAC). Thus, it is envisioned that the method of the invention is useful for developing an allelic profile of a subject for the TTR gene. “Allelic profile”, as used herein, is a determination of the composition of a subject's genome in regard to the presence or absence, and the copy number, of the TTR allele or variants thereof.

In one embodiment, the invention provides a method of determining predisposition of a subject to transthyretin amyloidosis or a disease or disorder associated therewith. The method includes determining the TTR allelic profile of a subject by isolating the nucleic acid specimen from the subject which includes the TTR sequence and determining the presence or absence of a mutation in the TTR nucleic acid sequence. The invention also provides a diagnostic or prognostic method for determining the TTR allelic profile of a subject including isolating a nucleic acid sample from the subject; amplifying the nucleic acid with ASO primers which hybridize to target sequences.

Several methods are available for detection of allelic variants in TTR. For example, allele specific oligonucleotides (ASO's) can be used as primers to identify such variants. ASO primers can be any length suitable for amplification of a target template nucleotide sequencing using a polymerase chain reaction assay. For example, in one embodiment ASO primers are 15-50 nucleotides in length and the 3′ nucleotide of at least one primer of a primer pair is specific for either the wild type TTR nucleotide or a specific TTR variant nucleotide. In one embodiment, the 3′ nucleotide of the forward primer of a primer pair is specific for either the wild type TTR nucleotide or a specific TTR variant nucleotide. In one embodiment, the 3′ nucleotide of the reverse primer of a primer pair is specific for either the wild type TTR nucleotide or a specific TTR variant nucleotide.

In one embodiment, ASO primers of the present invention can be used for allele-specific amplification of a fragment of TTR containing nucleotide 424 or nucleotide 148.

In one embodiment, ASO primers of the present invention can be used for allele-specific amplification of a fragment of TTR containing nucleotide 424. In one embodiment, the primers are for amplification of a wild-type allele containing a “G” at nucleotide 424. In one embodiment, the primers for amplification of the wild-type allele are SEQ ID NO:1 and SEQ ID NO:3. In one embodiment, the primers for amplification of the wild-type allele are SEQ ID NO:4 and SEQ ID NO:5. In one embodiment, the primers are for amplification of a variant allele containing an “A” at nucleotide 424. In one embodiment, the primers for amplification of the variant allele are SEQ ID NO:2 and SEQ ID NO:3. In one embodiment, the primers for amplification of the variant allele are SEQ ID NO:4 and SEQ ID NO:6.

### TTR Assay

When it is desirable to amplify the target nucleic acid sequence before detection, such as a TTR nucleic acid sequence, this can be accomplished using oligonucleotide(s) that are primers for amplification. These oligonucleotide primers are based upon identification of the flanking regions contiguous with the target nucleotide sequence. For example, in the case of TTR, these oligonucleotide primers comprise sequences which hybridize with nucleotide sequences flanking TTR exon 3.

The methods of the invention may also be used to diagnose a mammal with having or being at risk of developing transthyretin amyloidosis or a disease or disorder associated therewith, including, but not limited to, senile systemic amyloidosis (SSA), familial amyloid polyneuropathy (FAP), and familial amyloid cardiomyopathy (FAC). Thus, in certain embodiments, the method comprises contacting a target nucleic acid sample from the subject with at least one set of ASO primers, wherein the ASO primers are specific for a 424A variant of the TTR gene, amplifying the target nucleic acid sequence and detecting an amplified product.

In one embodiment, the method may be used to identify a subject as being either heterozygous or homozygous for a variant. In such an embodiment, the method comprises contacting a target nucleic acid sample from the subject with at least one set of ASO primers, wherein at least one primer is specific for a 424G wild type allele of the TTR gene, and at least one set of ASO primers, wherein at least one primer is specific for a 424A variant of the TTR gene, amplifying the target nucleic acid sequence and detecting an amplified product, wherein the presence of an amplified product from both sets of primers indicates that the subject is heterozygous at nucleotide 424 of the TTR gene, whereas the presence of an amplified product from a single set of primers indicates that the subject is homozygous. In one embodiment, the method further comprises diagnosing a subject as having an increased risk of developing transthyretin amyloidosis or a disease or disorder associated therewith, including, but are not limited to, senile systemic amyloidosis (SSA), familial amyloid polyneuropathy (FAP), and familial amyloid cardiomyopathy (FAC), based on the presence of an amplification product indicating that the subject is heterozygous or homozygous for the variant allele of the TTR gene.

In certain embodiments, the methods of the invention further comprise administering a treatment or therapeutic agent to the diagnosed subject. As used herein, the term “therapeutic agent” includes agents that provide a therapeutically desirable effect when administered to an animal (e.g., a mammal, such as a human). The agent may be of natural or synthetic origin. For example, it may be a nucleic acid, a polypeptide, a protein, a peptide, or an organic compound, such as a small molecule. The term “small molecule” includes organic molecules having a molecular weight of less than about, e.g., 1000 amu. In one embodiment a small molecule can have a molecular weight of less than about 800 amu. In another embodiment a small molecule can have a molecular weight of less than about 500 amu.

In certain embodiments, the treatment or therapeutic agent is salt restriction, loop diuretics (e.g., torsemide, bumetanide), aldosterone antagonists, angiotensin inhibitors, angiotensin receptor blockers, beta blockers, calcium channel blockers, digoxin, midodrine, and venous compression stockings. In certain embodiments, the treatment or therapeutic agent is an agent to treat or prevent a comorbid condition or complication of transthyretin amyloidosis, for example, polyneuropathy, carpal tunnel syndrome, autonomic insufficiency, and cardiomyopathy and gastrointestinal features, occasionally accompanied by vitreous opacities and renal insufficiency.

Therefore, in various embodiment, the methods of the invention may further comprise administering a therapeutic agent to subject (e.g., a patient identified as having an increased risk of transthyretin amyloidosis using a method described herein). Such a therapeutic agent may be formulated as pharmaceutical composition and administered to a subject, such as a human patient, in a variety of forms adapted to the chosen route of administration, e.g., orally or parenterally, by intravenous, intramuscular, topical or subcutaneous routes.

### Kits

The invention also includes a kit comprising at least one ASO primer of the invention and instructional material which describes, for instance, use of the at least one ASO primer for identifying a genetic variant in a sample. Such kits may contain, for example, various reagents for individual assays and instructions for their use. Typical kit components include, for example, (1) one or more ASO primers for amplification of specific variant alleles; (2) one or more ASO primers for amplification of specific reference alleles; and (3) reagents for PCR amplification.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, are not to be construed as limiting in any way the remainder of the disclosure.

### Example 1. Construction of Allele-Specific Oligonucleotides (ASO) Probes for Rapid PCR-Based Detection of TTR variant c.424G>A, p.Val142Ile (p.V122I)

A rapid, high-quality assay has been developed which delivers the genetic test results within less than 1 hour from blood “sample-in” to “results out”. To maximize clinical utility, this molecular genetic diagnostic test was developed to be run in less than 1 hour (see Table 3). This was possible because of 3 key features: 1) the assay works from blood. This removes the step of DNA extraction from blood which takes at least 1 hour. 2) Fast PCR, which is a commercially available instrument, and 3) the design of specific primers and optimized PCR conditions to work with the Fast PCR instrument.

Allele-specific oligonucleotide (ASO) primers were designed that are used in a rapid PCR instrument (35 cycles, <30 min) to generate specific amplification products for TTR variant c.424G>A, p.Val142Ile. The products are analyzed using rapid gel-electrophoresis to identify both the homozygous (wildtype or variant) or heterozygous TTR variant. To achieve high sensitivity and specificity in this test, two changes were engineered into the ASO primers. In each ASO primer, the third base from the 3′ end was mutated to increase allele-specificity, and phosphorothioate bond modifications were added to the last 3′ base to be able to use a high-fidelity DNA polymerase (with 3′-5′-exonuclease activity) in the PCR reaction. Assay conditions (e.g., PCR buffer, cycle conditions, Primer Melting Temperature (Tm)) were empirically optimized to be able to use human blood as the specimen type, which removes the DNA extraction step and shortens assay time. The assay has exceptional sensitivity and only uses a fraction of 1 drop of blood (<50 μl), which can be collected using a standard pinprick lancing device.

The test readout is simple: Negative, Positive Heterozygous, or Positive Homozygous. Heterozygous identifies risk for disease and need for family screening on one side of the family. Homozygous identifies risk for earlier and/or more severe disease and also identifies need for more extensive family screening.

The experimental methods and results are now described.

Development of ASO primers

Allele-specific oligonucleotides (ASO) were designed for detection of TTR variant c.424G>A, p.Val142Ile (FIG. 1). Multiple ASO for TTR V122I were designed in both the forward and reverse direction and at two different lengths (200 bp, 538 bp) (Table 1). This choice was made to enable detection of both homozygous (wildtype or variant) or heterozygous TTR variants, to create redundancy for primer failures, to enable primer multiplexing, and to increase assay speed. Primers were checked for hybridization specificity using BLAST against the human reference genome sequence. The third base from the 3′end of the ASO primer was mutated to increase the allele specificity of the oligonucleotide in both the WT and the variant oligonucleotide (Liu et al., 2012, Plant Methods, 8(1):34). In addition to this modification, phosphorothioate bond modifications were added to the last 3′ base of each ASO primer (allele specific base) (Di Gustio and King, 2003, Nucleic Acids Res, 31(3):e7). This choice was made to be able to use a high-fidelity DNA polymerase in the assay, which however has 3′-5′-exonuclease activity. Multiple different DNA polymerases were tested, both with or without exonuclease activity, and combinations of these different polymerases, and it was found that the assay required a DNA polymerase with 3′-5′-exonuclease activity.

Phosphorothioate bond substitutes a sulfur atom for a non-bridging oxygen in the phosphate backbone of an oligo. This modification renders the internucleotide linkage resistant to nuclease degradation. Phosphorothioate bonds can be introduced between the last 1-5 nucleotides at the 5′- or 3′-end of the oligo to inhibit exonuclease degradation. Including phosphorothioate bonds throughout the entire oligo will help reduce attack by endonucleases as well. ASO with multiple phosphorothioate bases were tested and it was found that some configurations worked well.

TABLE 1
Primers

| Primer | | Product | |
|---|---|---|---|
| Name | Primer Sequence | size | SEQ ID NO: |
| For WT | CTATTCCACCACGGCTGGC*G | 200 bp | 1 |
| For Mut | CTATTCCACCACGGCTGGC*A | 200 bp | 2 |
| Rev | AGTGGAATGAAAAGTGCCTTTCACAGGAA | 200 bp | 3 |
| For | TGGGAAGAATGTTTCCAGCTC | 538 bp | 4 |
| Rev WT | CATTCCTTGGGATTGGTTA*C | 538 bp | 5 |
| Rev Mut | CATTCCTTGGGATTGGTTA*T | 538 bp | 6 |

* = phosphorothioate bond
Underlined 3^{rd} base mutated

### TTR (p.V122I) Allele-Specific DNA Diagnostic Testing

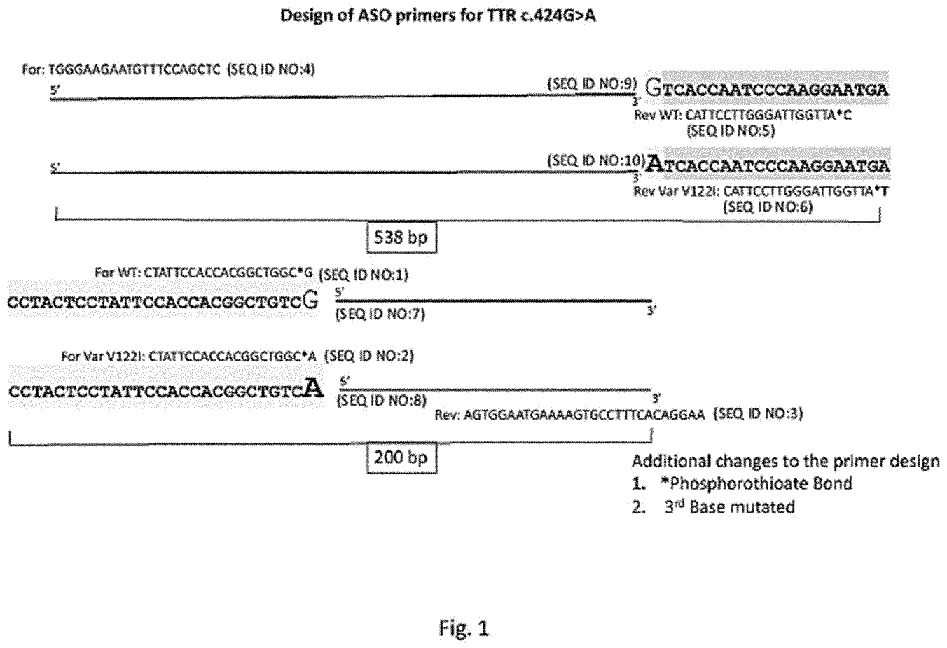

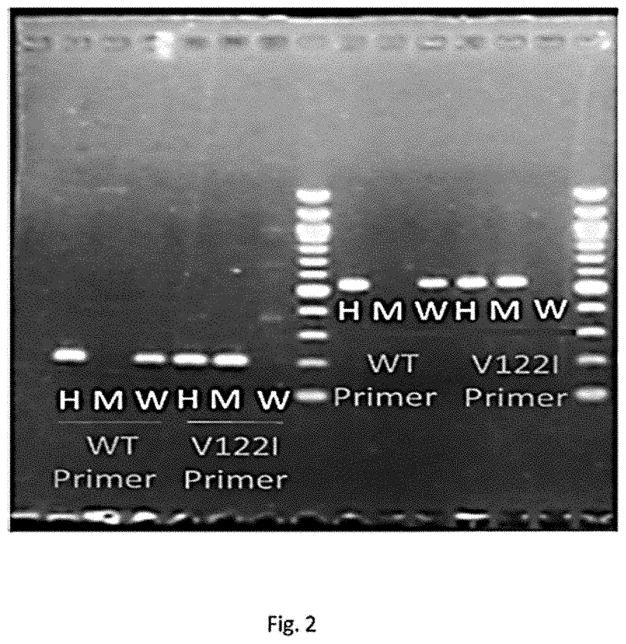

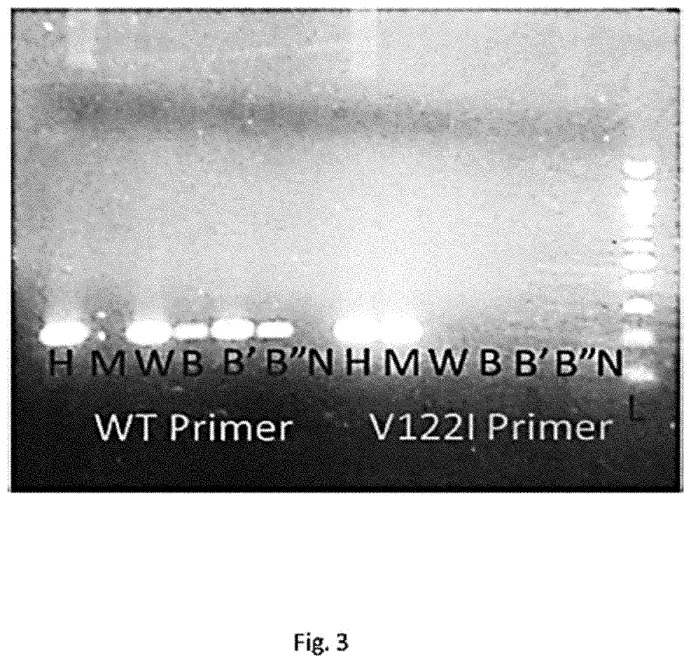

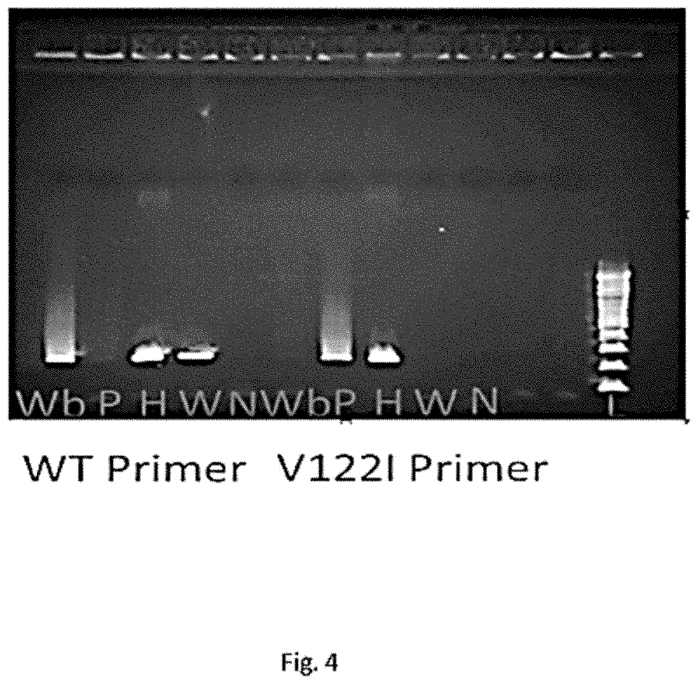

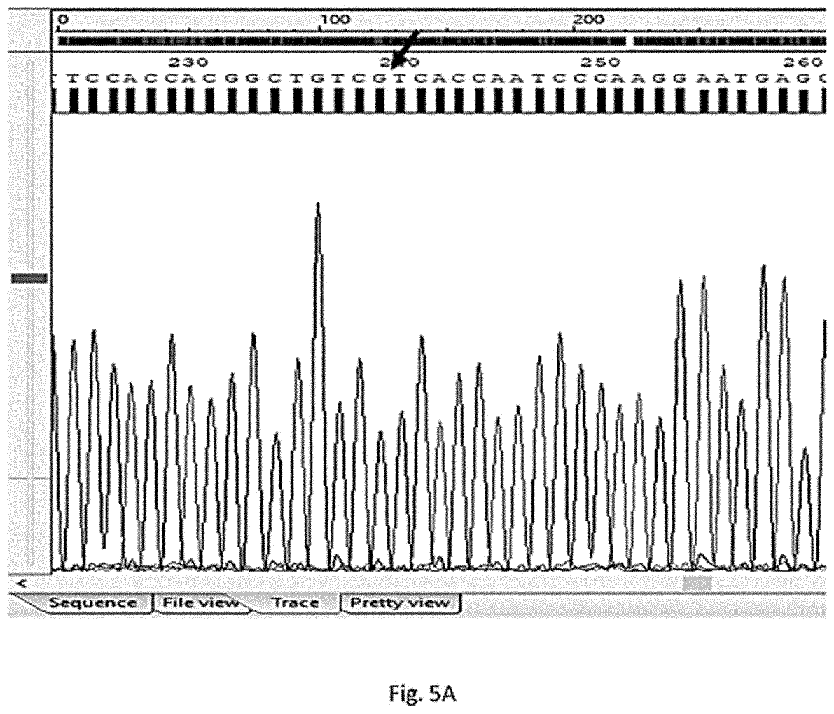

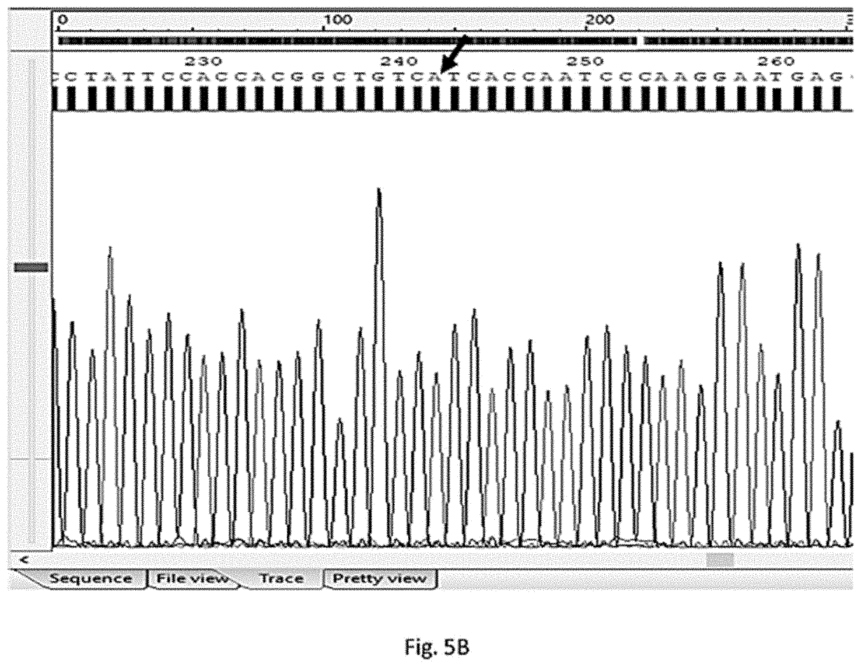

Using genomic DNA from 13 patients heterozygous and homzygous for V122I, the assay's allele-specific primers worked for both the TTR wildtype (reference sequence) and the V122I variant allele (FIG. 2 through FIG. 4). The data for the samples and primers is provided in Table 2. The assay conditions used are provided in Table 3. The TTR wildtype primers worked for pinprick blood. Pinprick blood from patients was required to test the V122I primer. Using a EDTA blood sample from 1 patient with confirmed c.424G>A, p.Val142Ile, the assay shows the patient is homozygous (no WT product). Sanger sequencing confirmed that the patient is homozygous for V122I variant (G>A) (FIG. 5).

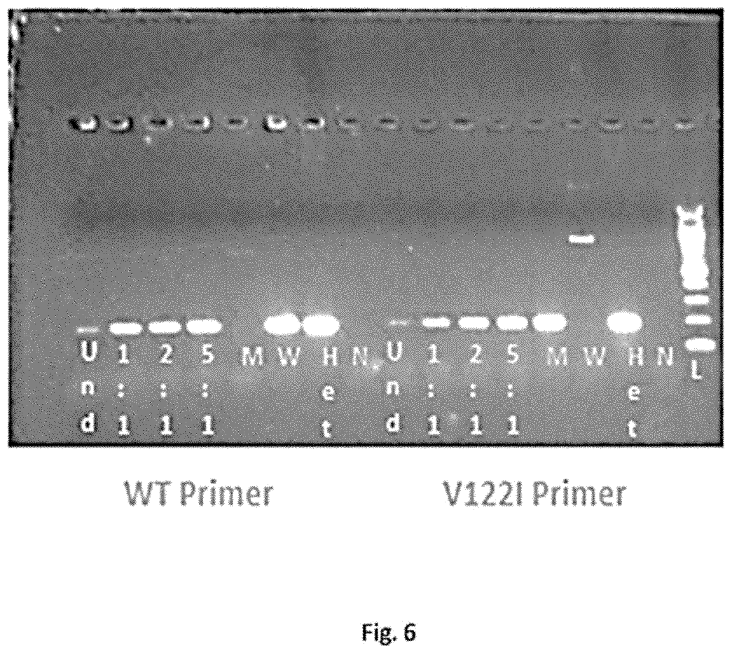

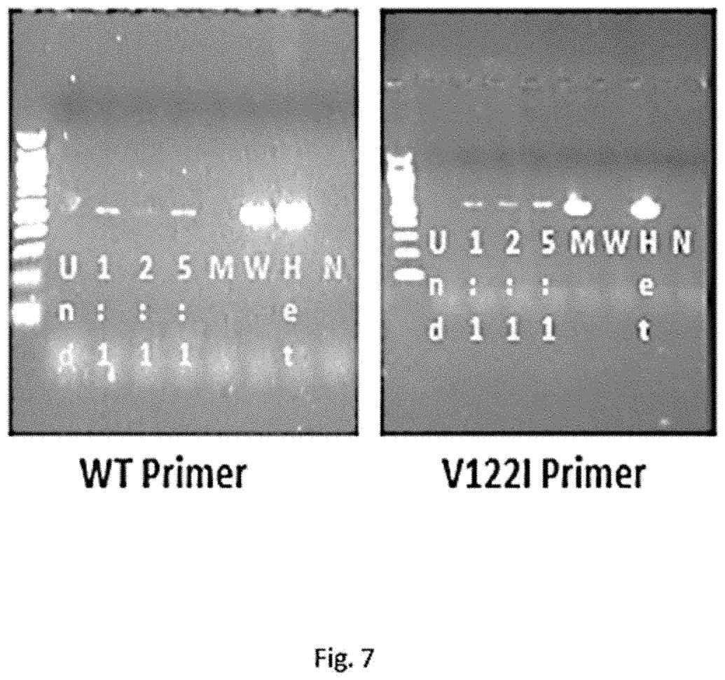

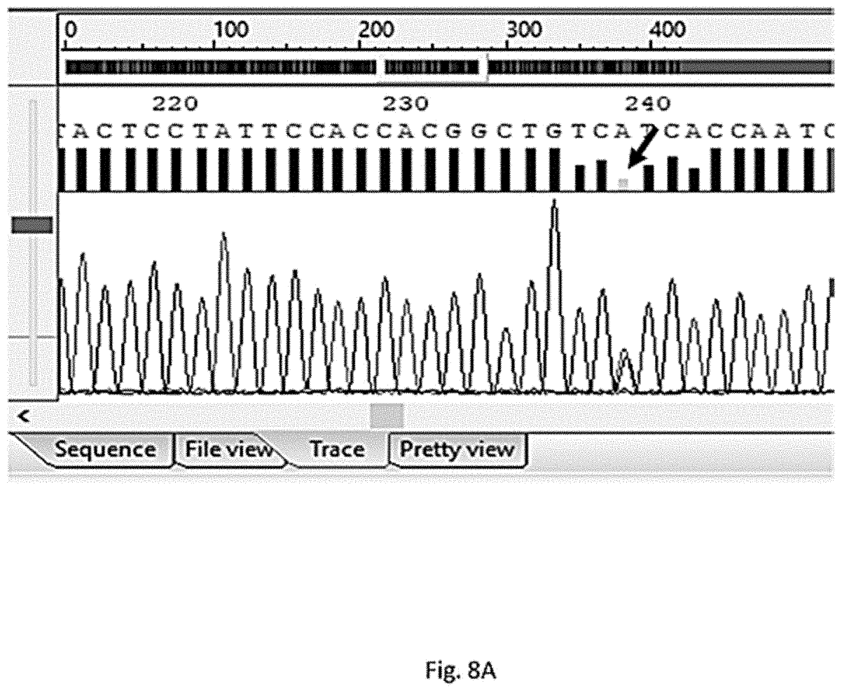

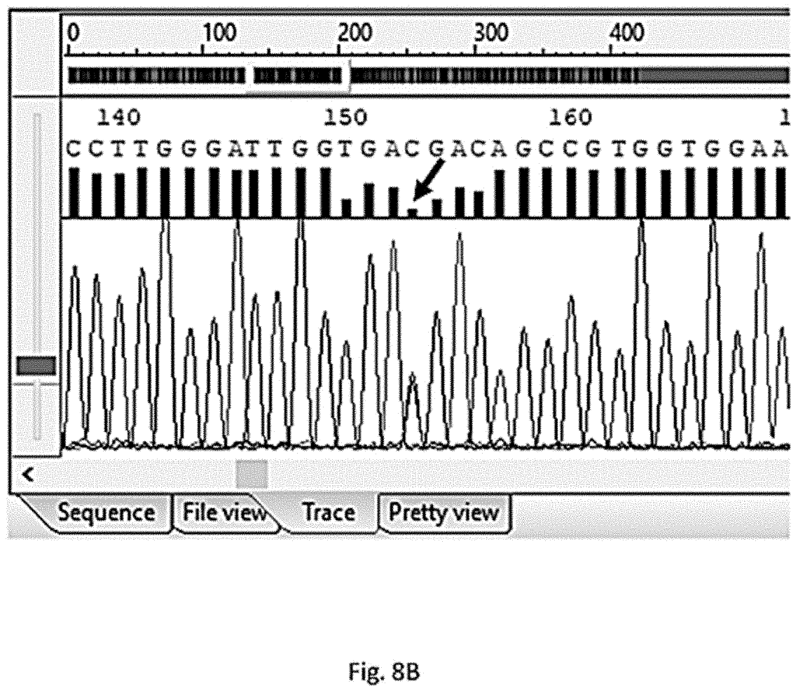

Next a Nik-2 patient sample was tested and identified as being heterozygous for the V122I variant (FIG. 6 through FIG. 8). This was confirmed with Nik regarding genotype.

TABLE 2
TTR (p. V122I) allele-specific DNA diagnostic testing

| | Samples | Interpretation |
|---|---|---|
| Genomic DNA | | |
| | H = V122I Heterozygous | WT primer [symbol] |
| | M = V122I Homozygous | V122I primer [symbol] |
| | W = Wildtype | |
| | Tested 13 DNA samples | |
| | (12 het, 1 hom) | |
| | Tested two primer pairs: | |
| | 200 bp product (left side) | |
| | 538 bp product (right side) | |
| | Ladder: 100 bp | |
| Pinprick (Genteel) | | |
| | H = V122I Het (DNA control) | WT primer [symbol] |
| | M = V122I Hom (DNA control) | V122I primer [symbol] |
| | W = Wildtype (DNA control) | |
| | N = Negative control (no DNA) | |
| | B = Fresh blood, undiluted | WT primer [symbol] |
| | B′ = Fresh, undil., frezze/thaw | V122I primer □ |
| | B″ = Fresh blood, diluted (1:1) | |
| | Tested 1 primer pair: 200 bp | □ not tested |
| Blood (EDTA) | | |
| | Wb = Wildtype blood (EDTA) | WT primer [symbol] |
| | P = Patient blood, V122I het | V122I primer [symbol] |
| | H = V122I Het (DNA control) | |
| | W = Wildtype (DNA control) | |
| | N = Negative control (no DNA) | |

TABLE 3
TTR Assay

| Protocol steps | Time | |
|---|---|---|
| Collect blood (Genteel) | 3 | minutes |
| Pre-PCR set up | 3 | minutes |
| NGPCR run | 8 | minutes (38 cycles) |
| Spin contents | 1 | minutes |
| Run Fast Gel | 5 | minutes |
| Interpretation | 2 | minutes |
| Total time: | 22 | minutes |

TABLE 4
Nik-2 Patent Sample

P = V122I Patient blood (EDTA) Nik-2 (Und = undiluted
blood, 1:1, 2:1, 5:1 dilution with water)
Controls:
W = WT blood (EDTA)
M = V122I homo patient blood (EDTA)
Het = Het DNA
N = Negative control
L = 100 bp Ladder

### Multiplex Assay

The design of primers for amplification of products with distinct sizes allows for combining multiple primers in a single PCR. For example, 200, 293 and 538 products could be generated in a single tube reaction by pooling all primers required to generate these products. The length difference between these products could be easily distinguished using gel electrophoresis. Multiplexing makes testing more efficient as multiple TTR loci (p.Val142Ile, p.Va150Met) could be tested in the same reaction.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A system comprising at least one set of primers for nucleic acid amplification, comprising:
a) at least one variant allele-specific primer, wherein the 3′ nucleotide of the variant allele-specific primer is selected from the group consisting of: a nucleotide that is the first allelic variant of a target sequence and a nucleotide that is complementary to the first allelic variant of a target sequence, wherein at least one nucleotide at a position selected from the group consisting of 5, 4, 3 and 2 bases from the 3′end of the primer is not complementary to the target sequence, and wherein the first allele-specific primer comprises at least one modified linkage; and
b) at least one locus-specific primer that is complementary to a region of the target sequence that is upstream or downstream from the first allelic variant and on the opposite strand.

2. The system of claim 1, wherein the nucleotide at the position 3 bases from the 3′end of the allele-specific primer is not complementary to the target sequence.

3. The system of claim 1, wherein the first allele-specific primer comprises at least one phosphorothioate linkage.

4. The system of claim 1, further comprising at least one reference allele-specific primer, wherein the 3′ nucleotide of the reference allele-specific primer is selected from the group consisting of: a nucleotide that is the wild-type or reference nucleotide of a target sequence of a target sequence and a nucleotide that is complementary to the wild-type or reference nucleotide of a target sequence, wherein at least one nucleotide at a position selected from the group consisting of 5, 4, 3 and 2 bases from the 3′end of the primer is not complementary to the target sequence, and wherein the reference allele-specific primer comprises at least one modified linkage.

5. The system of claim 4, wherein the nucleotide at the position 3 bases from the 3′ end of the reference allele-specific primer is not complementary to the target sequence.

6. The system of claim 4, wherein the reference allele-specific primer comprises at least one phosphorothioate linkage.

7. The system of claim 1, comprising at least two variant allele-specific primers, wherein the at least two variant allele-specific primers are specific for the same variant allele, wherein at least one variant allele-specific primer is a forward primer comprising a nucleotide that is the first allelic variant of a target sequence and at least one variant allele-specific primer is reverse primer comprising a nucleotide that is complementary to the first allelic variant of the target sequence; and at least two locus-specific primers.

8. The system of claim 1, comprising at least one allele-specific primer specific for a transthyretin 424G>A variant.

9. The system of claim 8, comprising at least one allele-specific primer selected from the group consisting of SEQ ID NO:2 and SEQ ID NO:6.

10. The system of claim 8, further comprising at least one reference allele-specific primer selected from the group consisting of SEQ ID NO:1 and SEQ ID NO:5.

11. The system of claim 8, comprising at least one locus specific primer selected from the group consisting of SEQ ID NO:3 and SEQ ID NO:4.

12. A method of identifying a subject as having a genetic variant allele comprising:
a) contacting a nucleic acid sample of the subject with the system of claim 1,
b) amplifying a target nucleotide sequence in the nucleic acid sample; and
c) detecting an amplification product.

13. The method of claim 12 comprising contacting the sample with at least two sets of primers in a single reaction, wherein amplification with the at least two sets of primers generates amplification products with distinct sizes.

14. The method of claim 12, wherein the sample is a blood sample.

15. A method of identifying a subject as being heterozygous or homozygous for a genetic variant allele comprising:
a) contacting a nucleic acid sample of the subject with the system of claim 4,
b) amplifying a target nucleotide sequence in the nucleic acid sample; and
c) detecting one or more amplification product, wherein detection of an amplification product upon amplification with at least one variant allele specific primer and detection of an amplification product upon amplification with at least one reference allele specific primer is an indicator that the subject is heterozygous for the variant allele, whereas detection of an amplification product upon amplification with at least one variant allele specific primer but not upon amplification with at least one reference allele specific primer is an indicator that the subject is homozygous for the variant allele.

16. The method of claim 15, wherein the genetic variant allele is a disease-associated variant allele.

17. The method of claim 16, wherein the genetic variant allele is a transthyretin 424G>A variant allele.

18. The method of claim 17, wherein the method further comprises diagnosing the subject as having an increased risk of developing a disease or disorder selected from the group consisting of transthyretin (TTR) amyloidosis, senile systemic amyloidosis (SSA), familial amyloid polyneuropathy (FAP), and familial amyloid cardiomyopathy (FAC) based upon identification that the subject is heterozygous or homozygous for the transthyretin 424G>A variant allele.

19. The method of claim 15 comprising contacting the sample with at least two sets of primers in a single reaction, wherein amplification with the at least two sets of primers generates amplification products with distinct sizes.

20. The method of claim 15, wherein the sample is a blood sample.
